(19) United States
(12) Patent Application Publication
(10) Pub. No.: US 2025/0157621 A1
(43) Pub. Date: May 15, 2025

(54) AUTOMATED SYSTEM AND METHOD FOR GENERATING A PRESCRIPTION FOR ORTHODONTIC APPLIANCES

(71) Applicant: Michael Wright, Clarence Center, NY (US)

(72) Inventor: Michael Wright, Clarence Center, NY (US)

(21) Appl. No.: 18/941,134

(22) Filed: Nov. 8, 2024

Related U.S. Application Data
(60) Provisional application No. 63/597,365, filed on Nov. 9, 2023.

(51) Int. Cl. G16H 20/40; A61C 7/00; B33Y 50/00; B33Y 80/00; G06T 7/00; G06T 19/20; G06V 10/776; G06V 20/64; G06V 40/10; G16H 80/00

(52) U.S. Cl. CPC G16H 20/40; A61C 7/002; B33Y 50/00; B33Y 80/00; G06T 7/0014; G06T 19/20; G06V 10/776; G06V 20/64; G06V 40/10; G16H 80/00; G06T 2200/24; G06T 2207/30036; G06T 2210/32; G06T 2210/41; G06T 2219/2004; G06V 2201/03

(57) ABSTRACT

A system and method for automatically generating orthodontic appliance prescriptions and designs, which utilizes a cloud-based platform integrated with technologies such as machine learning, natural language processing, and 3D printing. The system operates by receiving Standard Tessellation Language (STL) files from user devices, analyzing these files using STL logic, and comparing them against a comprehensive database of stored STL files. Orthodontic appliance prescriptions are generated incorporating user input and utilizing natural language processing to interpret textual data. Upon selection of a prescription, graphical 3D logic generates a visual representation of the appliance, which is then translated into a 3D-printable STL file optimized for medical-grade materials. The platform may employ feedback mechanisms and AI-enabled features for additional functionalities, such as billing, case tracking, and customer support, while ensuring compliance with healthcare regulations and quality assurance protocols for the production of orthodontic appliances.

### CROSS-REFERENCE TO RELATED APPLICATIONS

This application claims priority to U.S. Provisional Patent Application having Ser. No. 63/597,365, filed on Nov. 9, 2023, the entire disclosure of which is hereby incorporated herein by reference.

### BACKGROUND

Orthodontics is the practice of repositioning a patient's teeth to improve dental function and/or cosmetic appearance. Typically, with any treatment, an initial mold or scan of the patient's teeth is made. This mold or scan provides a model of the patient's teeth that the orthodontist uses to formulate a treatment strategy. The orthodontic practitioner creates a desired treatment plan by selecting components that apply gentle pressure to the teeth in certain directions. Over a period of time, the teeth tend to slowly shift toward the desired orthodontically correct positions along the dental arch. Once the teeth have been moved to the desired location and are held in place for a certain period of time, the body responds by adapting bone and tissue to maintain the teeth in the desired location.

The repositioning of the patient's teeth may be accomplished using a variety of orthodontic appliances, including fixed appliances, removable appliances, functional appliances and palatal expanders, using a variety of brackets, bands, archwires, lipbumpers, face bows, and other components known in the art. For example, one type of orthodontic treatment that is in widespread use comprises a set of orthodontic appliances along with an archwire. The appliances typically include a number of small, slotted brackets, each of which is mounted on a corresponding tooth along the dental arch. The archwire is received in the slot of each bracket and forms a track to guide the teeth toward desired positions. Usually, a set of appliances and an archwire are provided for both the upper and the lower dental arch of the patient, and treatment of both arches is carried out at the same time. Typically, after a period of time, the brackets are removed and a removable retainer is used to further assist in retaining the teeth in the desired location.

In practice, after the orthodontist has selected the desired treatment plan, the required orthodontic appliances are ordered from an orthodontic lab that specializes in manufacturing orthodontic appliances. The orthodontist typically handwrites a prescription on a diagram of an upper and lower dental arch and submits the prescription to the manufacturing lab. Alternatively, the orthodontist may use a prescription portal provided by the lab or use a universal system to submit the prescription. The dental appliances are then fabricated and provided to the orthodontist. This process can be time consuming and illegible handwriting and inaccurate drawings may lead to incorrect fabrication of the appliances.

As can be appreciated, it would be desirable to provide a method and system for facilitating the preparation of orthodontic appliance prescriptions, and in particular, to provide an automated system and method for facilitating the generation of prescriptions. Desirably, such a method would be easy to use and would generate consistently legible prescriptions adaptable for use with any type of orthodontic appliance and with any orthodontic manufacturing lab.

### FIELD

This invention is related to the field of orthodontics, and more particularly, relates to an automated system and method for designing and ordering orthodontic appliances for patients.

### SUMMARY

Accordingly, it is the subject of this invention to provide a comprehensive Artificial Intelligence (AI) enabled platform designed to facilitate and optimize the interaction between orthodontists and dental laboratories. The system is capable of receiving textual input from orthodontists, interpreting the input, generating precise dental appliance prescriptions, and creating 3D Standard Tessellation Language (STL) files for direct 3D printing of dental appliances.

### BRIEF DESCRIPTION OF THE DRAWINGS

FIG. 1 is a flowchart illustrating the process of using an STL Building Engine to create a digital working anatomy model for 3D printing and subsequent generation of orthodontic appliances.

FIG. 2 is a flowchart illustrating a computer-generated method for processing and analyzing STL data to generate and display a treatment prescription.

FIG. 3 is a flowchart illustrating the steps of a computer-generated method for generating orthodontic appliances, including receiving user input, generating a work order, and storing a new STL file in the database.

FIG. 4 is a flowchart illustrating the sequential steps of initializing a machine learning platform, processing STL data, and refining pattern recognition and prescription accuracy through iterative feedback loops.

FIG. 5 is a flowchart illustrating the steps of a computer-generated method for processing STL files within a database and selecting treatment files through iterative analysis and selection logic.

FIG. 6 is a flowchart illustrating a computer-generated method for processing STL files and generating treatment prescriptions, starting from receiving user input and ending with the presentation of the treatment prescription.

FIG. 7 is a flowchart illustrating the process of generating a 3D-printed model for an orthodontic appliance, beginning with receiving user input and progressing through steps such as initiating 3D printing logic, identifying anatomical patterns, calculating wire placement, and optimizing the model for medical-grade materials before final storage in an STL database.

FIG. 8 is a flowchart illustrating the steps involved in processing STL data using machine learning algorithms and natural language processing for generating orthodontic appliance recommendations.

FIG. 9 is a flowchart illustrating the process of generating a treatment prescription and selecting an orthodontic appliance based on STL files.

FIG. 10 is a flowchart illustrating a method for processing an STL file with AI-enhanced features and generating orthodontic appliance prescriptions, followed by visual representation and feedback integration.

### DETAILED DESCRIPTION

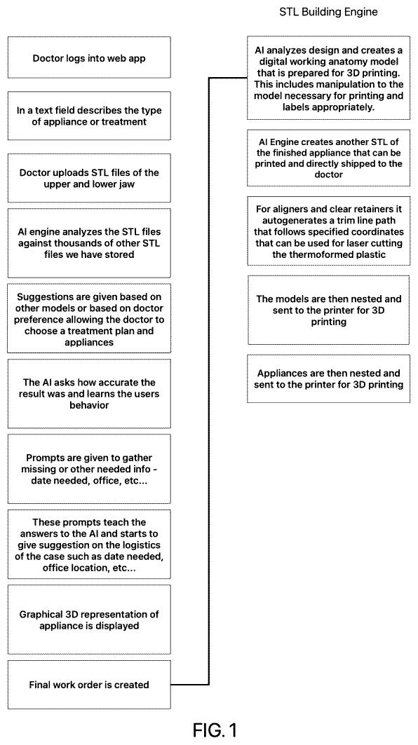

In one embodiment, the automated system and method for generating a prescription and design for orthodontic appliances is depicted in FIG. 1.

In one embodiment, the method includes the steps of:
receiving, by a cloud storage system, user input that includes an STL file from a device, wherein the cloud storage system includes STL logic for analyzing and comparing STL files;
responsive to receiving the STL file from the device, a cloud storage system server initiates STL logic to request analysis and comparison, by an STL API, of the STL file against other STL files stored in an STL database;
cloud storage system server initiates orthodontic appliance logic to generate, by an orthodontic appliance API, at least one orthodontic appliance prescription;
presenting, by the cloud storage system server, for display on the device the at least one orthodontic appliance prescription;
responsive to receiving, from a device, user input including a selected orthodontic appliance prescription, cloud storage system server initiates graphical 3D logic, by a graphical 3D logic API, thereby generating a final work order for an orthodontic appliance;
cloud storage system server initiates 3D printing logic, by a 3D printing API, thereby generating a 3D printing model;
cloud storage system server initiates STL logic to generate a new STL file of the 3d printing model; and,
responsive to receiving the new STL file, the STL database stores the new STL file.

In one embodiment, the STL database has at least 1,000 STL files.

In one embodiment, the system is provided with feedback so that it can learn user behavior and preferences.

In a preferred embodiment, the 3D-printed model is sent to a 3D printer for printing.

In some of the embodiments, the STL API, Orthodontic Appliance API, graphical 3D logic API, and 3D printing API, may be embodied as AI APIs.

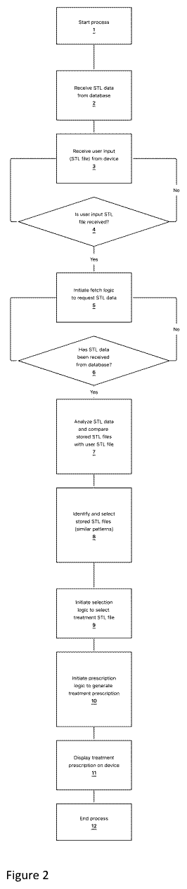

FIG. 2 shows a flowchart depicting a computer-generated method for processing STL data and generating orthodontic treatment prescriptions. The process initiates at step 1, marking the start of the process. Proceeding to step 2, STL data is received from a database, ensuring that foundational data is accessible for further processing.

At step 3, the system receives user input in the form of an STL file from a device. Step 4 involves a decision point where the system checks if the user input STL file has been successfully received. If confirmed, the process moves forward; otherwise, it loops back to await the file.

In step 5, the fetch logic is activated to request additional STL data, and the process advances to step 6, which confirms receipt of STL data from the database. Step 7 involves analyzing the STL data and comparing stored STL files with the user-provided STL file, focusing on identifying patterns.

Step 8 identifies and selects stored STL files that exhibit similar patterns, utilizing the STL logic to form a set of relevant files. Moving to step 9, the selection logic is initiated to choose a treatment STL file from the previously identified set.

In step 10, the system engages the prescription logic to generate a treatment prescription based on the selected STL file. The resulting treatment prescription is displayed on the device at step 11, providing the user with the necessary information. The process concludes at step 12, marking the end of the procedure.

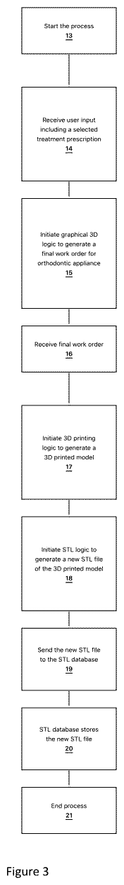

FIG. 3 shows a flowchart detailing the sequential steps involved in an automated process for generating orthodontic appliances. At step 13, the process is initiated. Step 14 involves receiving user input that includes a selected treatment prescription, facilitating personalized customization of the orthodontic appliance.

At step 15, graphical 3D logic is activated to produce a final work order for the orthodontic appliance. This step uses advanced graphical software to interpret the input data into a detailed design. In step 16, the final work order is received, confirming the specifications required for the next phase of production.

Continuing to step 17, the 3D printing logic is employed to fabricate the 3D printed model of the orthodontic appliance. This phase ensures the model is produced with precision suitable for practical application. Following this, step 18 initiates STL logic to create a new STL file of the completed 3D model, ensuring a digital record of the printed structure.

In step 19, the new STL file is transmitted to the STL database, methodically archiving the data. Step 20 involves the storage of this new STL file within the database, ensuring future accessibility and reference. Finally, at step 21, the process concludes, having successfully integrated design, production, and storage elements.

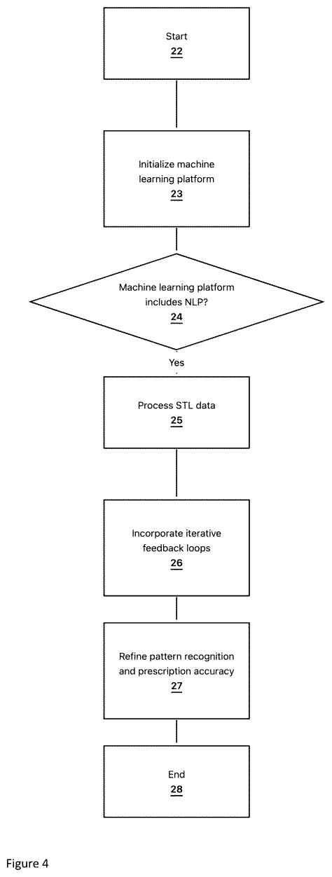

FIG. 4 shows a flowchart depicting a method for enhancing orthodontic appliance design using a machine learning platform. The sequence begins at step 22, where the process is initiated. Following this, step 23 involves the initialization of the machine learning platform, which serves as a foundation for subsequent data processing and analysis.

At step 24, the system checks whether the machine learning platform includes Natural Language Processing (NLP) capabilities. This decision point determines the pathway for processing data. If NLP is included, the pathway leads to step 25, where STL data is processed, enabling the system to handle and analyze the 3D models of dental structures.

The procedure continues with step 26, where iterative feedback loops are incorporated. This step allows the platform to continuously refine its algorithms, enhance pattern recognition, and improve prescription accuracy through accumulated data over time.

Lastly, step 27 focuses on refining pattern recognition and prescription accuracy, leveraging the feedback loops from the previous step. The method concludes at step 28, marking the end of the flowchart. Each step illustrates a distinct phase within the automated process, contributing to the overall enhancement of orthodontic appliance design and prescription generation.

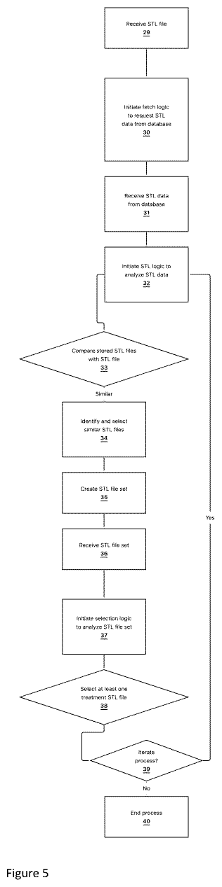

FIG. 5 shows a flowchart illustrating a method for processing STL files in an automated system for orthodontic appliance generation. At step 29, an STL file is received from a user device, initiating the sequence. Next, at step 30, fetch logic is initiated to request STL data from a database, ensuring that comprehensive data is available for analysis.

Step 31 involves receiving the STL data from the database. Subsequently, step 32 initiates STL logic to analyze this data. The comparison of stored STL files with the new STL file occurs at step 33, facilitating pattern recognition necessary for further processing.

At step 34, similar STL files are identified and selected based on the analysis. Following this, step 35 creates an STL file set comprising these selected files, which is then received at step 36 for further examination. At step 37, selection logic is initiated to analyze the STL file set, ensuring that only the most relevant files are utilized for prescription generation.

Step 38 involves selecting at least one treatment STL file from the analyzed set. The process then queries whether iteration is necessary at step 39, which, if so, loops back to repeat certain steps for refinement. If iteration is not required, the method concludes at step 40, ending the process.

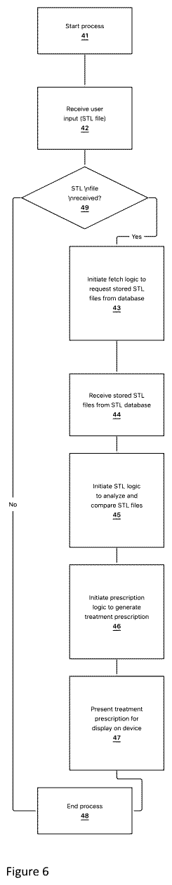

FIG. 6 shows a process flowchart depicting a method for generating orthodontic appliance prescriptions. The process begins at step 41 with the initiation of the method. At step 42, user input is received, specifically an STL file representing a 3D model of dental structures.

Following the reception of user input, step 49 determines whether the STL file has been successfully received. If the file is received, the process continues to step 43, where fetch logic is initiated to request stored STL files from a database.

At step 44, the system receives these stored STL files from the STL database. This is followed by step 45, where the system initiates STL logic to analyze and compare the received STL files against the stored files, utilizing comparative analysis to identify similar patterns.

Upon completion of the analysis, step 46 involves initiating prescription logic to generate a treatment prescription based on the analyzed data. The treatment prescription is then presented at step 47 for display on a user device, allowing for review and potential selection by the user.

The process concludes at step 48, where the process is ended, marking the completion of the method for generating and presenting orthodontic appliance prescriptions.

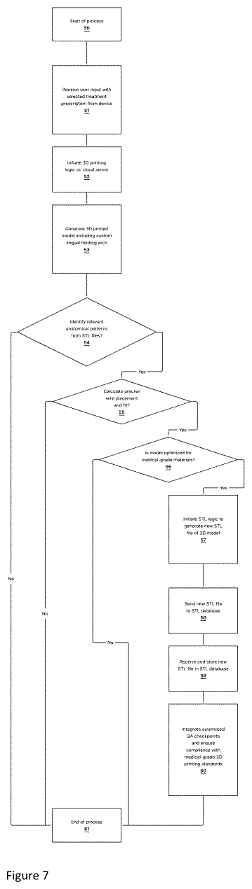

FIG. 7 shows a flowchart detailing an automated process for generating a 3D-printed orthodontic appliance model. The process begins with step 50, marking the start of the procedure. At step 51, user input is received, which includes a selected treatment prescription from a device. This input triggers step 52, where 3D printing logic is initiated on a cloud server.

Proceeding to step 53, a 3D printed model, including a custom lingual holding arch, is generated. At step 54, the system identifies relevant anatomical patterns from STL files. If patterns are identified, the process moves to step 55, where precise wire placement and fit are calculated. Step 56 evaluates whether the model is optimized for medical-grade materials. If optimization is confirmed, the procedure advances to step 57, where STL logic is initiated to generate a new STL file of the 3D model.

In step 58, the new STL file is sent to the STL database. Upon receipt, as depicted in step 59, the new STL file is stored within the database. Conclusively, step 60 integrates automated quality assurance checkpoints and ensures compliance with medical-grade 3D printing standards. The process concludes at step 61. If any evaluation steps such as steps 54, 55, or 56 are negative, the system loops back to previous steps as indicated by the flowchart, ensuring thorough validation before conclusion.

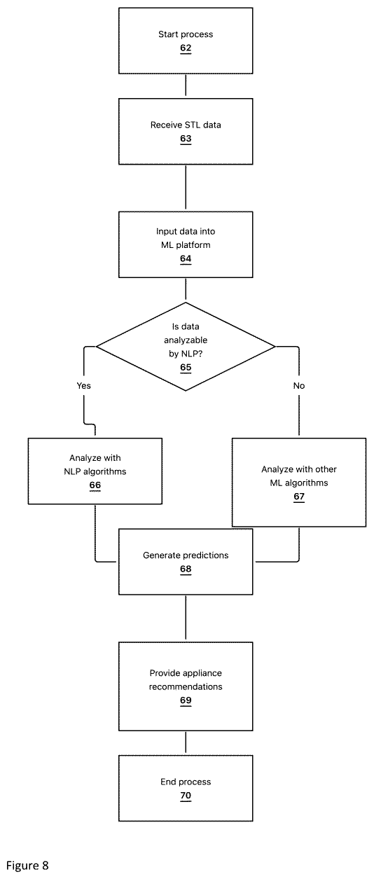

FIG. 8 shows a flowchart detailing a method for processing STL data related to orthodontic appliance generation. At step 62, the process initiates with starting logic to commence the overall operation. This introductory stage sets the foundation for the subsequent data reception and analysis.

Proceeding to step 63, the system receives STL data, which is crucial for analyzing three-dimensional dental structures. The data is then input into a machine learning (ML) platform at step 64, indicating the integration of advanced computational models to process and interpret the data effectively.

At step 65, a decision point determines whether the data is suitable for analysis using Natural Language Processing (NLP). If analyzable by NLP, the procedure advances to step 66, where NLP algorithms are employed for detailed analysis. Conversely, if NLP is not applicable, step 67 involves the utilization of alternate ML algorithms to extract meaningful insights from the data.

Upon completion of the analysis, step 68 generates predictions based on the processed data, potentially indicating orthodontic treatment paths or appliance options. Subsequently, at step 69, the system provides appliance recommendations, translating the analyzed data into actionable outputs for clinical application.

Finally, step 70 concludes the process, effectively terminating the sequence of operations and marking the end of the automated workflow for orthodontic appliance design generation. This structured approach demonstrates an integration of AI-driven technologies within a cloud-based platform, optimizing the design and creation of dental appliances.

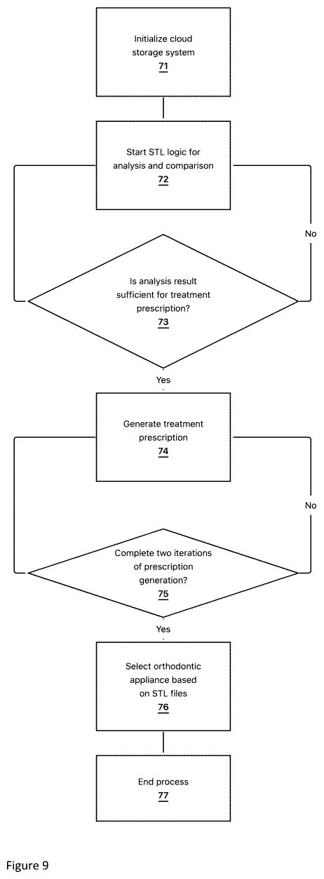

FIG. 9 shows a flowchart detailing a method for generating treatment prescriptions for orthodontic appliances utilizing a cloud-based system. At step 71, the process begins with the initialization of the cloud storage system, setting the groundwork for receiving and storing STL files necessary for subsequent analysis. Following initialization, step 72 involves the activation of STL logic designed for the analysis and comparison of incoming STL data against existing records in the system.

The process proceeds with step 73, where the results of the STL analysis are evaluated to determine if they are sufficient for generating a treatment prescription. If the evaluation indicates adequacy (Yes), the method advances to step 74, where the system generates a treatment prescription based on the analyzed data. Conversely, if the analysis is insufficient, the flow returns to step 72 for additional iterations, ensuring reliable prescription generation.

Step 75 acts as a decision point to confirm the completion of two iterations of prescription generation. Should this condition be met (Yes), the process continues to step 76. Here, an orthodontic appliance selection is conducted based on the STL files and treatment prescriptions generated in prior steps. The method culminates at step 77, marking the end of the process, encapsulating the systematic approach for orthodontic appliance design and prescription generation. This structured flow supports adaptability for various input scenarios and emphasizes precision in appliance customizations.

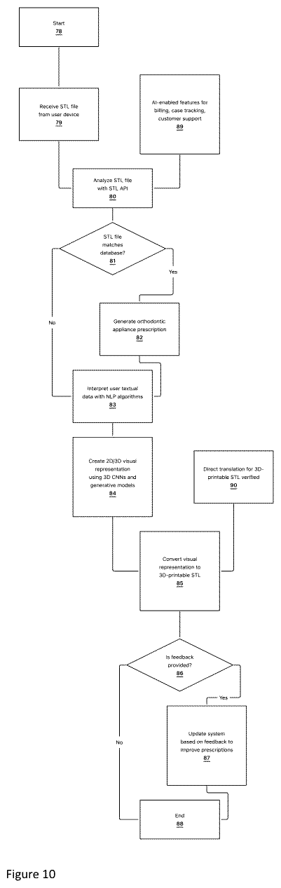

FIG. 10 shows a flowchart depicting a method for generating orthodontic appliance designs, utilizing a cloud-based platform integrated with various technologies. At step 78, the process begins. In step 79, the system receives an STL file from a user device, initiating the automated process.

At step 80, the STL file is analyzed using an STL API to determine if it matches any files within the database, leading to a decision point at step 81. If a match is identified, the process moves to step 82, where an orthodontic appliance prescription is generated. Concurrently, step 83 involves interpreting user textual data with natural language processing (NLP) algorithms for enhanced prescription accuracy.

The creation of a 2D or 3D visual representation is carried out in step 84, employing 3D Convolutional Neural Networks and generative models to render the design. This visual is converted to a 3D-printable STL file at step 85, enabling direct translation for production at step 90.

Step 89 outlines AI-enabled features that assist with billing, case tracking, and customer support, optimizing overall user experience. The decision point at step 86 determines whether feedback is provided; if so, the system updates at step 87 to refine future prescriptions based on this feedback. The method concludes at step 88, marking the end of the process.

### Benefits and Functions of the Platform:

### Textual Prescription Interpretation

Orthodontists can input textual descriptions specifying the type, characteristics, and requirements of a dental appliance. The AI uses Natural Language Processing (NLP) algorithms to interpret this data and create a detailed prescription that is comprehensible by dental labs.

### Machine-Learned Treatment Suggestions

Based on historical data and machine learning algorithms, the platform offers alternative treatment suggestions, presenting orthodontists with various appliance options that have shown success in similar cases.

### Visual and 3D Representation

The platform uses Generative Models and possibly 3D Convolutional Neural Networks to produce both 2D and 3D visual representations of the dental appliance, as described in the prescription. The orthodontists have the option to review, approve, or modify these visual representations.

### Direct-to-Print 3D Models

In a groundbreaking feature, the platform translates the approved 3D visual representation into a 3D-printable STL file. This file is optimized for medical-grade materials and is ready for direct 3D printing, thereby significantly reducing the lead time from prescription to appliance production.

### All-Inclusive AI Assistant

The platform is not limited to prescription and appliance design. It includes integrated features for billing, case status tracking, and customer support, which are also AI-enabled for efficiency and user-friendliness.

### Regulatory and Quality Assurance

The platform integrates quality assurance checkpoints and ensures compliance with healthcare regulations by incorporating standards for medical-grade 3D printing materials and processes.

By creating an end-to-end system that streamlines the process from prescription to appliance production, this invention aims to revolutionize the field of orthodontics by increasing efficiency, reducing error rates, and improving patient outcomes.

### EXAMPLES

### Example 1

Example 1, on the front end, an orthodontist will log into a web app and uploads or otherwise inserts a description of the type of appliance or treatment required for a particular patient. The orthodontist will also include Standard Tessellation Language (STL) files of the upper and lower jaw of the patient. The system will analyze and compare the STL file with other STL files stored in a database. The system will then use the STL files to generate several options for the orthodontist to select. Once the STL file is selected, the system will create a final work order and 3D printing model. The system will also create another STL file to store in the database. Finally, the 3D printing model is sent to a 3D printer for printing.

### Example 2

AI-Enabled Platform for Orthodontic Appliance Design and Production. This example describes an AI-powered platform to revolutionize how orthodontists design and produce dental appliances. By automating the entire process—from diagnosis to 3D printing—this platform eliminates inefficiencies, reduces errors, and enhances patient outcomes. As the platform evolves, it will leverage machine learning to predict and recommend the optimal appliance based solely on patient 3D scans (STL files), removing the need for orthodontists to manually select treatments.

### Detailed Workflow and Functionality

### Orthodontist Login and Case Submission:

Orthodontists log into a secure web-based platform and upload Standard Tessellation Language (STL) files containing 3D scans of the patient's upper and lower jaws. Initially, orthodontists input textual descriptions specifying appliance type and customizations, though this step will eventually be phased out as the AI becomes more proficient.

AI-Powered Appliance Recognition from STL Files:

Over time, the platform will learn to identify the required appliance from the STL scan, bypassing manual selection. By analyzing patterns and appliance characteristics across thousands of cases, the AI will discern the appropriate treatment based on the patient's dental structure alone.

### NLP-Driven Prescription Interpretation:

For early users, Natural Language Processing (NLP) algorithms convert textual input into a structured, digital prescription for lab technicians. The system interprets characteristics such as wire thickness, appliance type, and treatment specifics, ensuring accuracy and consistency.

Machine-Learned Treatment Suggestions: The platform uses historical data and machine-learning algorithms to recommend treatment options. Orthodontists receive appliance suggestions that align with their preferences and past successful cases, with options to modify and approve.

### 3D Visualization and Design Review:

Generative Models and 3D Convolutional Neural Networks (CNNs) generate 2D and 3D models of the appliance for orthodontists to inspect. Real-time adjustments are possible, allowing doctors to tweak the design before approval.

### Direct-to-Print 3D Model Generation:

Once approved, the platform generates STL files optimized for 3D printing using medical-grade materials. The system automatically generates trim-line paths for laser cutting for appliances like clear aligners.

### Automated Production and Case Logistics:

STL files are sent directly to a 3D printer for production, significantly reducing lead times. AI-powered logistics tracks each case, providing real-time updates on production and delivery.

### Integrated AI Assistant and Support:

The platform offers AI-enabled billing, case tracking, and customer support to streamline operations. Orthodontists receive proactive notifications about their cases, ensuring smooth communication and delivery.

### Regulatory Compliance and Quality Control:

Automated quality assurance (QA) checkpoints validate each design, ensuring compliance with medical-grade standards. The system's compliance with healthcare regulations guarantees safe and effective orthodontic appliances.

### Key Innovation: Predictive Appliance Selection Using STL Files

A groundbreaking feature of this platform is its ability to learn the appropriate appliance directly from STL scans. As more cases are processed, the system will develop a pattern recognition model capable of associating specific dental structures with optimal appliance types. Orthodontists will no longer need to select an appliance manually; rather the AI system will provide recommendations based entirely on the scans and case history. This evolution will reduce cognitive load for orthodontists and enhance treatment accuracy by relying on objective, data-driven insights.

### Example Use Case: Fully Automated Retainer Production

An orthodontist uploads patient STL scans; the AI system recognizes the need for a clear retainer based solely on the dental arches; the orthodontist reviews the AI-generated 3D model and approves it, the system generates the STL file, sends it to the 3D printer, and provides real-time case tracking.

### Benefits

Increased Efficiency: Automates tedious processes, speeding up production and reducing lead times.

Error Reduction: Eliminates manual errors and ensures consistent, accurate prescriptions.

Enhanced Customization: Machine learning allows the system to tailor recommendations to the orthodontist's preferences over time.

Cutting-Edge Predictive Capability: Predicts and recommends the appropriate appliance using only STL data, setting a new standard in orthodontics.

Regulatory Compliance: Adheres to industry standards, ensuring the safety and effectiveness of all appliances.

Improved Patient Outcomes: Faster, precise appliances improve results and patient satisfaction.

End-to-End Automation: The entire process is automated from prescription to production, minimizing delays and errors.

It will be appreciated by those skilled in the art that while the automated system and method for generating a prescription and design for orthodontic appliances has been described in detail herein, the invention is not necessarily so limited and other examples, embodiments, uses, modifications, and departures from the embodiments, examples, uses, and modifications may be made without departing from the process and all such embodiments are intended to be within the scope and spirit of the appended claims.

1. A computer generated method comprising the steps of:
a. receiving, by a database, STL data that includes stored STL files;
b. receiving, by a server or processor, user input that includes an STL file from a device, wherein the server or the processor includes fetch logic, STL logic for autonomously identifying patterns and performing comparative analysis on stored STL files, selection logic, and prescription logic;
c. responsive to receiving an STL file, the server or the processor initiates the fetch logic to request the STL data from the database;
d. responsive to receiving STL data from the database, the server or the processor initiates the STL logic to analyze the STL data and compare the stored STL files with the STL file;
e. identifying and selecting, by the the STL logic, stored STL files from the STL data that are similar to the STL file thereby generating an STL file set based on recognized dental structure patterns;
f. responsive to receiving the STL file set, the server or processor initiates the selection logic to analyze the STL file set and select at least one treatment STL file from the STL file set; and
g. responsive to receiving the at least one treatment STL file, the server or the processor initiates the prescription logic to generate at least one treatment prescription for display on the device.

2. The method of claim 1 further comprising the step: responsive to receiving, from the device, user input including a selected treatment prescription, the server or the processor initiates graphical 3D logic thereby generating a final work order for an orthodontic appliance.

3. The method of claim 2 further comprising the step: responsive to receiving a final work order, the server or the processor initiates 3D printing logic thereby generating a 3D printed model.

4. The method of claim 3 further comprising the step of: the server or the processor initiates STL logic to generate a new STL file of the 3D printed model and sends the new STL file to the STL database.

5. The method of claim 4 further comprising the step of: responsive to receiving the new STL file, the STL database stores the new STL file.

6. The method of claim 5, wherein the STL logic, selection logic, and prescription logic comprise a machine learning platform, wherein the machine learning platform incorporates iterative feedback loops, allowing it to refine pattern recognition and prescription accuracy with each use, based on historical STL data and newly acquired new STL files.

7. The method of claim 6, wherein the machine learning platform comprises natural language processing.

8. The method of claim 1, wherein the steps of responsive to receiving an STL file, the server or the processor initiates the fetch logic to request the STL data from the database; responsive to receiving STL data from the database, the server or the processor initiates the STL logic to analyze the STL data and compare the stored STL files with the STL file; identifying and selecting, by the the STL logic, stored STL files from the STL data that are similar to the STL file thereby creating an STL file set; and responsive to receiving the STL file set, the server or processor initiates the selection logic to analyze the STL file set and select at least one treatment STL file from the STL file set, are iterative and may be performed at least twice.

9. A computer generated method comprising the steps of:
a. receiving, by a cloud storage system, user input that includes an STL file from a device, wherein the cloud storage system includes fetch logic, STL logic for analyzing and comparing STL files and prescription logic for generating a treatment prescription;
b. responsive to receiving the STL file from the device, the cloud storage system initiates the fetch logic to request stored STL files from an STL database;
c. responsive to receiving the stored STL files from the STL database, the cloud storage system initiates the STL logic to request analysis and comparison of the STL file against the stored STL files;
d. the cloud storage system server initiates the prescription logic to generate at least one treatment prescription based on the results of the analysis and comparison performed by the STL logic; and
e. presenting, by the cloud storage system, for display on the device the at least one treatment prescription.

10. The method of claim 9 further comprising the step: responsive to receiving, from the device, user input including a selected treatment prescription, the cloud storage system server initiates 3D printing logic thereby generating a 3D printed model, wherein the 3D printed model includes a custom lingual holding arch as a result of the 3D printing logic identifying relevant anatomical patterns from STL files, calculating precise wire placement and fit, and wherein the 3D-printed model is optimized for medical-grade materials.

11. The method of claim 10 further comprising the step of: the cloud storage system initiates the STL logic to generate a new STL file of the 3D printed model and sends the new STL file to the STL database.

12. The method of claim 11 further comprising the step of: responsive to receiving the new STL file, the STL database stores the new STL file, and wherein the cloud storage system is capable of system integrating automated quality assurance checkpoints throughout the design process and is capable of ensuring compliance with medical-grade 3D printing standards and healthcare regulations.

13. The method of claim 9, wherein the STL logic and prescription logic comprise a machine learning platform and wherein the process includes predictive algorithms to recommend optimal appliances based on the STL data, providing orthodontists with suggestions without requiring manual selection.

14. The method of claim 13, wherein the machine learning platform comprises natural language processing.

15. The computer-generated method of claim 9, wherein the step of the cloud storage system initiates prescription logic to generate at least one treatment prescription based on the results of the analysis and comparison performed by the STL logic may be iterative and may be performed at least two times, wherein the cloud storage system is capable of autonomously selecting the appropriate orthodontic appliance based solely on STL files.

16. An automated system for generating orthodontic appliance designs, comprising:
a. a cloud-based platform configured to receive a Standard Tessellation Language (STL) file from a user device, the STL file representing a 3D model of a patient's dental structure;
b. a storage system including an STL database with at least 1,000 STL files, wherein the platform employs STL logic to analyze and compare the received STL file against files in the STL database via an STL API;
c. Orthodontic Appliance APIs, and Graphical 3D Logic APIs, each incorporating machine learning algorithms to enhance prescription generation and design recommendations;
d. orthodontic appliance logic configured to produce one or more orthodontic appliance prescriptions based on the analysis, utilizing Natural Language Processing algorithms to interpret textual data provided by a user;
e. graphical 3D logic configured to generate a 3D visual representation of a selected orthodontic appliance prescription, employing generative models and 3D Convolutional Neural Networks to create both 2D and 3D visual representations; and
f. a translation module to convert approved 3D visual representations into 3D-printable STL files optimized for medical-grade materials.

17. The system of claim 16, further comprising a feedback mechanism configured to learn from user behavior and preferences to improve prescription accuracy over time.

18. The system of claim 17, wherein the cloud-based platform includes AI-enabled features for billing, case tracking, and customer support, optimizing operations and ensuring seamless communication.

19. The system of claim 18, wherein the translation module is confirmed to directly translate the 3D visual representation into 3D-printable STL files.
